(19) United States
(12) Patent Application Publication
(10) Pub. No.: US 2008/0251083 A1
(43) Pub. Date: Oct. 16, 2008

(54) Tracheotomy kit and method

(76) Inventors: Richard M. Fetcenko, Cortland, OH (US); Michael E. Francis, Beaver Falls, PA (US); Michael J. Roche, Cortland, OH (US); Daniel J. Erdman, West Middlesex, PA (US); Steven W. Marzo, Cortland, OH (US); Todd W. McCune, Chesapeake, VA (US)

Correspondence Address: DELPHI TECHNOLOGIES, INC., M/C 480-410-202, PO BOX 5052, TROY, MI, 48007, US

(21) Appl. No.: 11/787,271

(22) Filed: Apr. 16, 2007

(51) Int. Cl. A61M 16/00

(52) U.S. Cl. 128/207.29

(57) ABSTRACT

A tracheotomy kit has a cutter assembly and an end cap. The cutter assembly includes a cutter head protruding from an end of a cutter housing that has flexible arms that extend from opposite sides of the cutter housing forwardly and outwardly in cantilever fashion to a position forward of a tip of the cutter head. The free ends of the flexible arms have pads for engaging a human throat for performing a tracheotomy. The end cap is detachably fastened to the cutter housing enclosing the cutter head and includes a flat elongate tongue for spreading the tracheotomy incision.

### BACKGROUND OF THE INVENTION

This invention relates generally to tracheotomies and more particularly to a tracheotomy kit and method for performing a tracheotomy.

The conventional method to perform a tracheotomy today uses a hand held scalpel or other sharp blade to cut through the throat skin and trachea of a patient by hand. The size and depth of the incision is operator sensitive. Moreover the skill necessary to perform a tracheotomy by hand without causing further injury is not possessed by the average layman nor even by most paramedics and military medics. Consequently, performing an emergency tracheotomy, for instance to an injured soldier in the battle field is avoided often due to the lack of skill. On the other hand, avoiding the emergency tracheotomy, prior to transport to an aid station, or other place where the skill exists, may be catastrophic even to the point of resulting in death. Consequently there is a need for a tracheotomy kit and method that can be used by anyone to perform a tracheotomy with little or minimal training.

### SUMMARY OF THE INVENTION

This invention provides a tracheotomy kit and method that allows anyone, such as an average layman, a paramedic or a military medic, to perform a tracheotomy with little or minimal training. Thus the tracheotomy can be performed in any emergency situation, such as at the scene of an accident or on the battlefield, thereby reducing the risk of serious injury and possible death due to delay or an unskilled tracheotomy.

### BRIEF DESCRIPTION OF THE DRAWINGS

FIG. 1 is a perspective view of a tracheotomy kit that embodies the invention;

FIG. 2 is a exploded perspective view showing an end cap and a cutter assembly, of the tracheotomy kit shown in FIG. 1, in different perspectives;

FIG. 3 is a top perspective view of a sub-assembly of the cutter assembly shown in FIG. 2;

FIG. 4 is a bottom view of the cutter assembly shown in FIG. 2;

FIG. 5 is a side view of the cutter assembly shown in FIG. 2 in place on a neck of a patient for performing a tracheotomy,

FIGS. 6 and 7 are front views of the cutter assembly shown in FIG. 2 in the process of performing a tracheotomy;

FIGS. 8, 9 and 10 are side views of the end cap in the process of performing a follow-up procedure; and

FIGS. 11 and 12 are perspective views of further embodiments of a cutter assembly for the tracheotomy kit shown in FIG. 1.

### DETAILED DESCRIPTION OF THE PREFERRED EMBODIMENTS

Referring now to the drawings, tracheotomy kit 10 comprises a cutter assembly 11 having a housing base 12, a cutter head indicated generally at 14 and a housing cover 16. An end cap 18 is detachably connected to the cutter assembly 11.

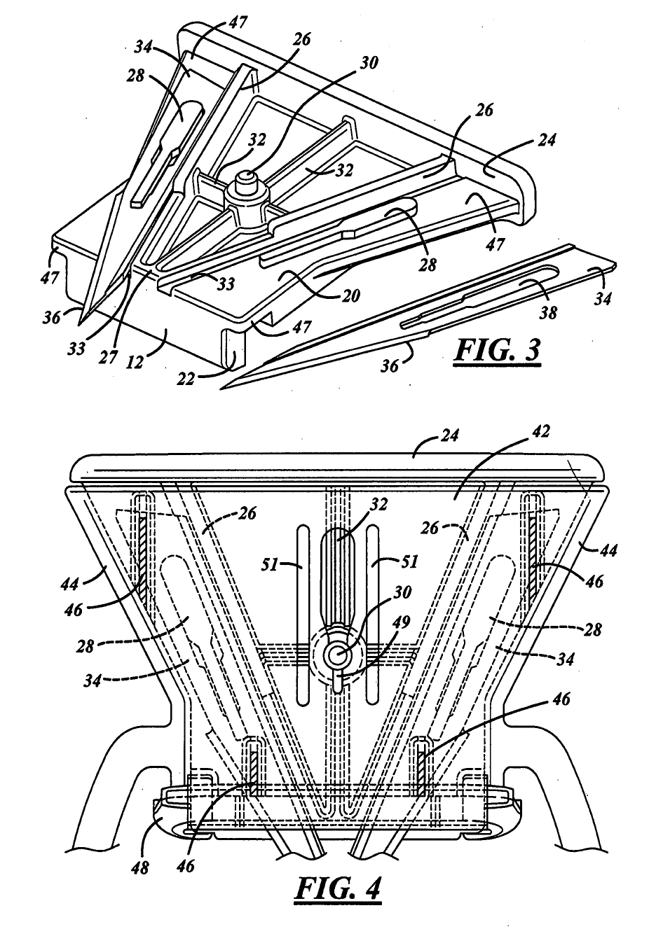

Housing base 12 has a generally flat body 20 comprising a forward rectangular portion that terminates with a forward down flange 22, and a rearward trapezoidal portion that terminates with a rearward up flange 24 as best shown in FIG. 3. The flat body 20 has two raised guide rails 26 that extend from the rearward up flange 24 toward the forward end of the housing base 12. Guide rails 26 converge toward each other in the forward direction and are connected by a short lateral rib 27 at the forward end of the housing base 12.

The generally flat body 20 also has two raised, shorter, paddle shaped retention ribs 28 outward of the respective guide rails 26 and a raised lock nib 30. The raised lock nib 30 is located between the guide rails 26 and extends above the guide rails 26. The generally flat body 20 may also include strengthening ribs 32 inside guide rails 26 and a shallow groove 33 adjacent the outer side of each of the guide rails 26.

Cutter head 14 is preferably flat and wedge shaped as best shown in FIG. 2. Cutter head 14 may be provided by two flat blades 34, such as standard scalpel blades, that have pointed cutting edges 36 and paddle shaped cut-outs 38 as best shown in FIG. 3. Blades 34 are retained on the generally flat body 20 of housing base 12 by the paddle shaped retention ribs 28 projecting into the paddle shaped cut-outs 38. Blades 34 are located by the retention ribs 28 engaging cut-outs 38 and the raised rails 26 engaging the respective inner sides of the blades 34 so that the pointed cutting edges 36 that project outwardly of the forward end of the body 20 converge toward each other to provide the flat wedge shaped cutter head 14.

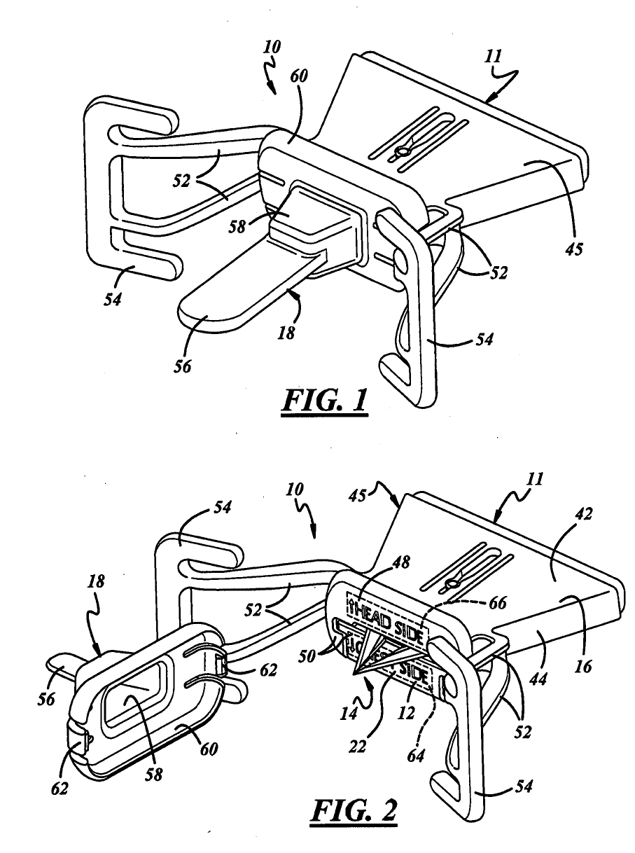

Housing cover 16 comprises a generally flat body 42 having a forward rectangular portion, a rearward trapezoidal portion, and side flanges 44 that engage respective sides of housing base 12 to form a cutter housing 45 for the flat blades 34. Four hold down ribs 46 project down from the generally flat body 42 into the cutter housing 45. The side flanges 44 have channels that receive respective side wings 47 of housing base 12 so that the housing cover 16 is held down on the housing base 12 and the hold down ribs 46 engage and clamp the flat blades 34 down against the housing base 12 as best shown in FIG. 4. Housing cover 16 is retained on housing base 12 longitudinally by lock nib 30 engaging a flexible socket portion of a longitudinal lock slot 49 in the flat body 42 as shown in FIGS. 1, 2 and 4. The flexibility of the socket portion may be enhanced by a longitudinal slot 51 on each side of the lock slot 49.

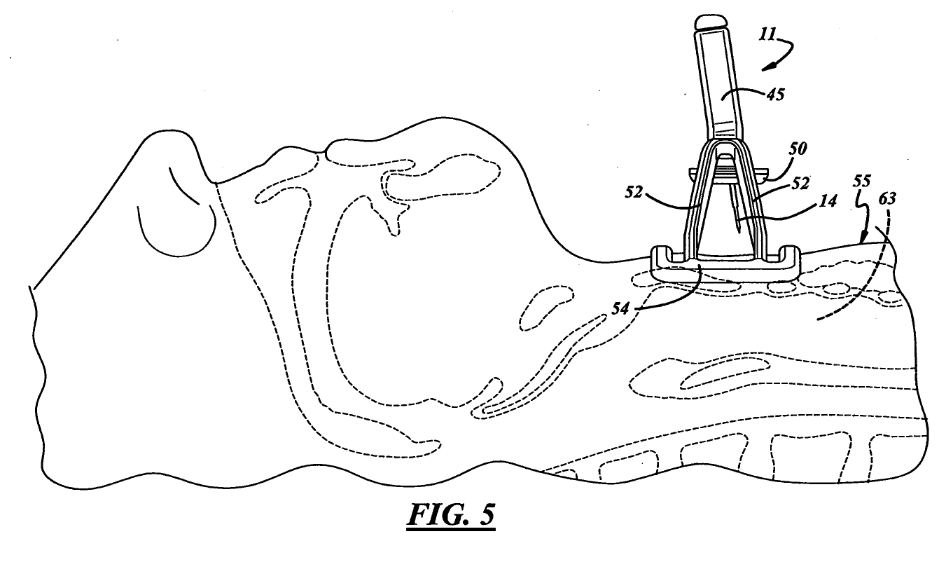

Housing cover 16 has a forward flange 48 that mates with the forward down flange 22 of the housing base 12 to provide an enlarged stop wall 50 at the forward end of cutter housing 45 as best shown in FIG. 2. Housing cover 16 also has two flexible arms 52 that are attached to the respective side flanges 44 of the housing cover 16 and that project forwardly and outwardly of the stop wall 50 in cantilever fashion to respective free ends that are forward of the wedge shaped cutting head 14 as best shown in FIGS. 2 and 5. The flexible arms 52 which may be bifurcated for enhanced flexibility have enlarged U-shaped pads 54 at their respective free ends. The U-shaped pads 54 diverge and are shaped for engaging a human throat 55 as shown in FIGS. 5-7. Moreover, the flexible arms 52 and pads 54 are preferably shaped to orient the cutter housing 45 and the projecting flat, wedge shaped cutter head 14 at an angle of about 8 to 12 degrees from perpendicular to the neck as indicated in FIG. 5.

End cap 18 has a thin flat, elongate tongue 56 at one end and a small trapezoidal cup 58 at an opposite end that terminates in an enlarged mounting hood 60. Hood 60 is detachably fastened to the enlarged stop wall 50 by lock tabs 62 so that the trapezoidal cup 58 covers the projecting cutter head 14 as shown in FIG. 1. When the end cap 18 is attached to stop wall 50 of the housing 45, tongue 56 preferably projects forwardly of the pads 54 at the respective ends of the flexible arms 52 as best shown in FIG. 1. This facilitates detachment of the end cap 18.

In the event that an emergency tracheotomy is required, such as in a battlefield, the tracheotomy kit 10 can be used by a minimally trained military medic to provide an opening in the throat of a wounded soldier in a controlled manner to allow the insertion of a breathing tube. First, the end cap 18 is detached to expose cutter head 14 as shown in FIG. 2. End cap 18 may be detached easily by pushing the outer flat end of the elongate tongue 56 laterally.

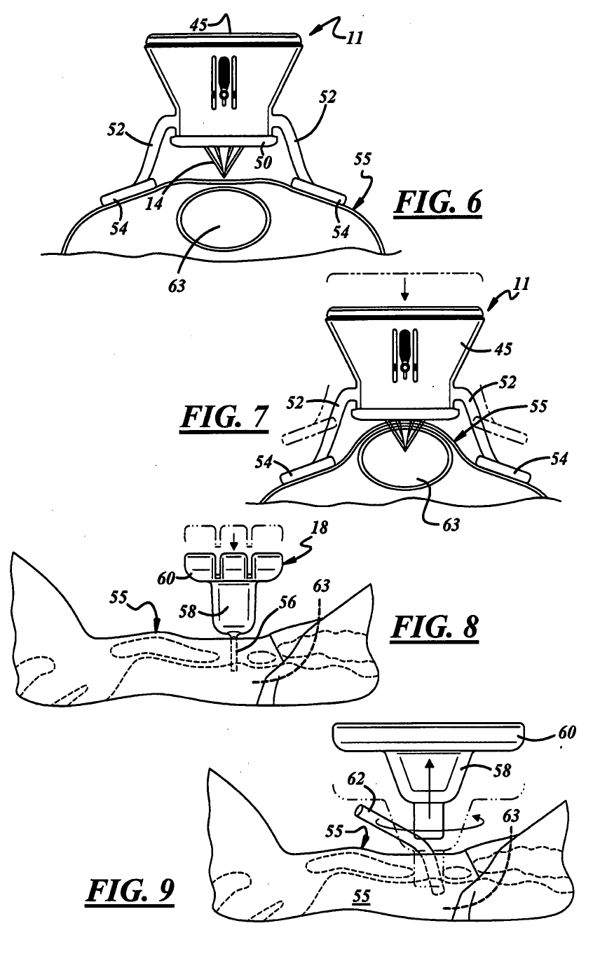

The cutter assembly 11 is then positioned on the wounded soldiers throat 55 between the larynx (voice box) and the sternum (breastbone) where the opening is to be made as shown in FIGS. 5 and 6. The two flat blades 34 oppose each other and preferably meet at the points with the sharp edges outward to form a double edged cutter head 14. The angle at which blades 34 are set relative to each other and the amount of blade projection from the stop wall 50 of the cutter housing 45 control the width and depth of the cut into the throat 55. The two enlarged U-shaped pads 54 of the flexible arms 52 that protrude forward and outward of the cutter head 14 contact the throat 55 before the cutter head 14 engages the throat as shown in FIG. 6. The flexible arms 52 and U-shaped pads 54 align the cutter housing 45 and cutter head 14 at an angle to the throat 55 to assure the cutting blades 34 of the cutter head 14 are slanted away the head to avoid any possible damage to the larynx (voice box) during the cutting process. This angle is preferably in the range of 8 to 12 degrees and more preferably is about 10 degrees.

The cutter housing 45 is then simply pushed down until the enlarge stop wall 50 engages throat 55 and the cutter head 14 cuts through the skin and into the trachea 63 as shown in FIG. 7. As cutter housing 45 is pushed down, the flexible arms 52 bend outwardly spreading the U-shaped pads 54 apart. The spreading U-shaped pads 54 spread the skin beneath the cutter housing 45 taut to improve the cutting process by the double edged cutter head 14. It should be noted that the wide U-shaped pads 54 at the ends of the flexible arms 52 accommodate various neck sizes. Moreover, the broad flat stop wall 50 at the forward end of the cutter housing 45 assures that the double edged cutting head 14 penetrates the front of trachea 63 only and does not pass through the back side of the trachea.

It is estimated that it takes a force of about 5 to 15 pounds to push the cutter housing down to make the incision. The cutter housing 45 may be pushed down with the palm of the hand easily because the trapezoidal shaped rearward portion of the cutter housing 45 provides a relatively wide flange 24 for pushing the cutter housing down 45 down as well as accommodating the angled cutter blades 34 inside the cutter housing 45. The cutter housing 45 can also be formed with other ergonomic friendly features for better control under battlefield or other hazardous conditions, for example, finger and thumb pads or a generally thicker body for easier gripping and/or texturized non-slip surfaces.

The end cap 18 of the tracheotomy kit 10 protects the cutter head 14 and kit handlers while attached. After detachment, the end cap 18 also accommodates a follow-up procedure for the tracheotomy as shown in FIGS. 8, 9 and 10. After cutting through the throat tissue and front of the trachea 63, cutter assembly 11 is withdrawn. The flat elongate tongue 56 of end cap 18 is then inserted into the incision through the throat tissue and the front of the trachea 63 as shown in FIG. 8.

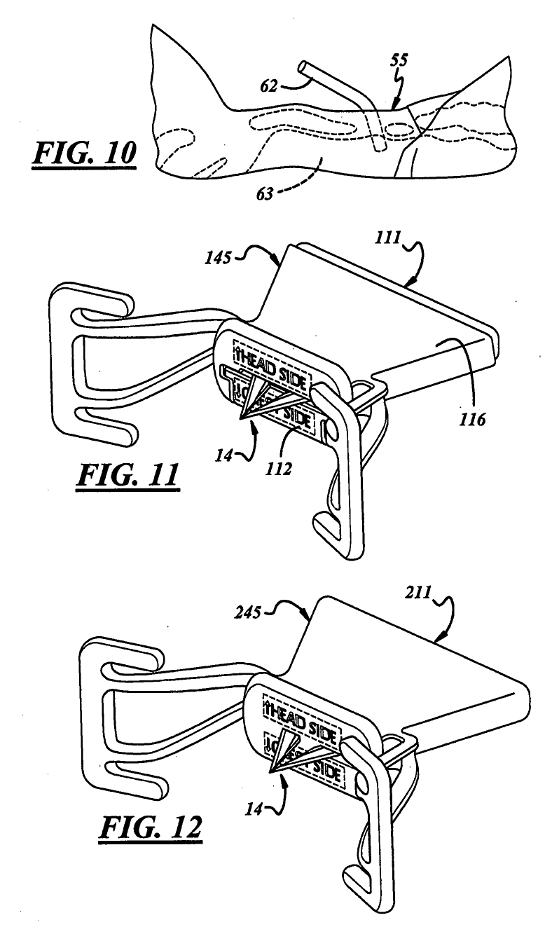

End cap 18 is then rotated approximately 90° creating a larger opening for the insertion of a breathing tube 62 as shown in FIG. 9. End cap 18 is then withdrawn allowing the throat 55 to close around the breathing tube 62 as shown in FIG. 10.

After use the cutter assembly 11 can be disassembled, the cutter housing components and cutter blades sterilized and the cutter assembly 11 reassembled. (The cutter blades 34 may be replaced with new blades if desired.) It should be noted that the two part cutter housing 45 does not require any tools for disassembly because the slide lock formed by lock nib 30 and lock slot 47 allows for housing cover 16 to be detached and reattached by hand. If new blades 34 are desired, conventional scalpel blades 34 may be used and held in the desired position by retention ribs 28 in the housing base 12 that match the standard slot of the conventional scalpel blades 34. Hold down ribs 46 on the housing cover 16 apply force against the blades 34 to prevent excessive blade movement once the blade housing 45 is reassembled.

Proper orientation of the blade housing 45 on the throat relative to the head and chest of the patient may be accomplished by markings on the cutter housing 45 indicating which side faces the head and which side faces the chest of the patient. For instance, the housing base 12 could be marked with an arrow and “chest side” and the housing cover with an arrow and “head side” as shown by optional labels at 64 and 66 in FIG. 2.

As indicated above, the flat blades 34 are held at an angle towards the chest when the cutter housing 45 is located on the throat by the flexible arms 52 and U-shaped pads 54 adding additional protection against injuring the voice box of the patient. A thin flat elongate blade 56 is integrated into the end cap 18 to spread the incision made in the throat and trachea for insertion of a temporary breathing tube.

The cutter housing 45 is preferably molded in two parts from a suitable plastic such as nylon or other medical grade plastic. While the housing base 12 and housing cover 16 can be detached and reattached manually, the housing base and housing cover can be modified for permanent attachment if a one-time-use, disposable cutter assembly is desired. For example, the housing cover 116 can be permanently attached to the cover base 112 by welding, gluing or otherwise permanently securing the two parts together to form a cutter housing 145 for cutter assembly 111 as shown in FIG. 11. Moreover, it is also possible to insert mold the two blades 34 or a single properly shaped blade in a one-piece cutter housing 245 for a cutter assembly 211 as shown in FIG. 12. Either cutter assembly 111 or 211 may be used as a disposable cutter assembly in place of cutter assembly 11 in the tracheotomy kit 10.

In other words, it will be readily understood by those persons skilled in the art that the present invention is susceptible of broad utility and application. Many embodiments and adaptations of the present invention other than those described above, as well as many variations, modifications and equivalent arrangements, will be apparent from or reasonably suggested by the present invention and the foregoing description, without departing from the substance or scope of the present invention. Accordingly, while the present invention has been described herein in detail in relation to its preferred embodiment, it is to be understood that this disclosure is only illustrative and exemplary of the present invention and is made merely for purposes of providing a full and enabling disclosure of the invention. The foregoing disclosure is not intended or to be construed to limit the present invention or otherwise to exclude any such other embodiments, adaptations, variations, modifications and equivalent arrangements, the present invention being limited only by the following claims and the equivalents thereof.

We claim:

1. A tracheotomy kit comprising;
a cutter assembly having a cutter head protruding from an end of a cutter housing,
the cutter housing having flexible arms that extend from opposite sides of the cutter housing forwardly and outwardly in cantilever fashion to a position forward of a tip of the cutter head, and
the flexible arms having free ends shaped for engaging a human throat for performing a tracheotomy.

2. The tracheotomy kit as defined in claim 1 wherein the tracheotomy kit further includes an end cap that is fastened to the cutter housing and encloses the cutter head protruding from the end of the cutter housing.

3. A tracheotomy kit comprising;
a cutter assembly having a wedge shaped cutter head protruding from an enlarged stop wall at an end of a cutter housing,
the cutter housing having flexible arms that extend from opposite sides of the cutter housing forwardly and outwardly in cantilever fashion to a position forward of a tip of the cutter head, and
the flexible arms having pads at their free ends that are shaped for engaging a human throat for performing a tracheotomy.

4. The tracheotomy kit as defined in claim 3 wherein the tracheotomy kit further includes an end cap that is detachably fastened to the cutter housing and encloses the cutter head protruding from the end of the cutter housing, the end cap having a flat elongate tongue at a free end.

5. The tracheotomy kit as defined in claim 3 wherein the cutter housing has a housing base and a housing cover that is detachably connected to the housing base, and wherein the cutter head is part of at least one cutter blade having a portion that is retained in the cutter housing between the housing base and the housing cover.

6. The tracheotomy kit as defined in claim 3 wherein the cutter housing has a housing base and a housing cover that is permanently connected to the housing base, and wherein the cutter head is part of at least one cutter blade having a portion that is retained in the cutter housing between the housing base and the housing cover.

7. The tracheotomy kit as defined in claim 3 wherein the cutter housing has a housing base and an integral housing cover, and wherein the cutter head is part of at least one cutter blade having a portion that is insert molded in the cutter housing.

8. The tracheotomy kit as defined in claim 4 wherein the cutter housing has a housing base and a housing cover that is detachably connected to the housing base, and wherein the cutter head is part of two cutter blades having portions that are retained in the cutter housing between the housing base and the housing cover.

9. A tracheotomy kit comprising;
a cutter assembly having a cutter housing and a wedge shaped cutter head protruding from an enlarged stop wall at a forward end of the cutter housing,
the cutter housing having a housing base and a housing cover,
the housing base having a generally flat body having a forward rectangular end portion and a rearward trapezoidal end portion terminating in a rearward up flange, the forward rectangular portion terminating in a forward down flange forming part of the enlarged stop wall at the forward end of the cutter housing, the generally flat body of the housing base having first and second raised guide rails that extend from the rearward up flange toward the forward end and converge toward each other, first and second raised retention ribs located outward of the first and second raised guide rails respectively, and a raised lock nib disposed between the first and second raised guide rails,
the housing cover having a generally flat body having a forward rectangular portion, a rearward trapezoidal portion, hold down ribs, and side flanges engaging respective sides of the housing base, the side flanges of the housing cover having channels that receive respective side wings of the housing base, and the housing cover having a forward flange that aligns with the forward flange of the housing base to form the enlarged stop wall,
the cutter head being part of first and second flat blades having portions that are retained on the housing base by the first and second retention ribs respectively, the first and second flat blades having pointed cutting edges that project outwardly of the forward end of the housing base and converge toward each other to form the wedge shaped cutter head, and
flexible arms that are attached to the respective side flanges of the housing cover and that project forwardly and outwardly of the enlarged stop wall in cantilever fashion to respective free ends that are forward of the wedge shaped cutter head, the flexible arms having U-shaped pads at their respective free ends.

10. The tracheotomy kit as defined in claim 9 wherein the tracheotomy kit further includes an end cap that is detachably fastened to the cutter housing, the end cap having a flat elongate tongue at one end and a trapezoidal housing at another end enclosing the cutter head protruding from the end of the cutter housing, and the trapezoidal housing terminating in a enlarged mounting flange that is detachably connected to the enlarged stop wall of the cutter housing.

11. A method of performing a tracheotomy using a tracheotomy kit comprising a cutter assembly having a cutter head protruding from an end of a cutter housing, the cutter housing having flexible arms that extend from opposite sides of the cutter housing forwardly and outwardly in cantilever fashion to a position forward of a tip of the cutter head, and the flexible arms having free ends shaped for engaging a human throat for performing a tracheotomy comprising the steps of;
placing the cutter assembly on the throat of a person with the free ends of the flexible arms engaging the throat between the larynx and the sternum of the person, and
pushing the cutter housing down until the cutter head penetrates through the throat skin and a front portion of the trachea to make an incision through the throat skin and the front portion of the trachea.

12. The method of claim 10 wherein the cutter head is pushed down until the end of the cutter housing engages the throat skin of the person.

13. The method of claim 12 wherein the tracheotomy kit includes an end cap that is attached to the cutter housing and encloses the cutter head protruding from the end of the cutter housing and wherein the end cap is removed before the cutter assembly is placed on the throat of the person.

14. The method of claim 13 wherein the end cap has a flat elongate blade, wherein the cutter housing is withdrawn from the throat after the incision is made and wherein the flat elongate blade is inserted into the incision and twisted to prepare the incision for insertion of a breathing tube.
